(19) United States
(12) Patent Application Publication
(10) Pub. No.: US 2002/0016535 A1
(43) Pub. Date: Feb. 7, 2002

(54) Subcutaneous glucose measurement device

(76) Inventors: W. Blake Martin, Birmingham, AL (US); Micah D. Schmidt, Burtonville, MD (US)

Correspondence Address: Jason A. Bernstein, BERNSTEIN & ASSOCIATES, P.C., Embassy Row 400, Suite 495, 6600 Peachtree Dunwoody Road, N.E., Atlanta, GA, 30328-1649, US

(21) Appl. No.: 09/770,131

(22) Filed: Jan. 26, 2001

Related U.S. Application Data
(60) Provisional application No. 60/178,596, filed on Jan. 28, 2000.

(51) Int. Cl. A61B 5/00

(52) U.S. Cl. 600/319

(57) ABSTRACT

A subcutaneous glucose sensor comprising an infrared emitter which transmits light through a narrow sample of interstitial fluid held in a light transparent sample trough. The sensor can be incorporated with an insulin pump in order to create an insulin delivery and feedback measurement loop system.

### CROSS-REFERENCE TO RELATED APPLICATION

The present application claims benefit of copending provisional patent application Ser. No. 60/178,596, filed Jan. 28, 2000, which is incorporated by reference herein.

### FIELD OF THE INVENTION

The present invention relates generally to medical sensors, and, more particularly, to glucose sensors for implanting in the human body.

### BACKGROUND OF THE INVENTION

Glucose sensing technology is needed in medical applications primarily to monitor the condition of patients with diabetes mellitus. This disease inhibits the body's ability to metabolize glucose. This inability to metabolize glucose can be due to a lack of the hormone insulin, which allows glucose to be transported into somatic cells. The disease may also be due to a defect that causes the insulin produced by the body to be inefficient. The current recommended treatment for this disease involves tight regulation of the patient's diet, monitoring of blood sugar levels, and injection of insulin or ingestion of sugar when needed. Widely fluctuating blood sugar levels may lead to acute damage, diabetic shock, and death in severe cases. More mild fluctuations in blood sugar levels can lead to long term complications such as blindness, loss of circulation and sensation in the extremities heart disease, and kidney failure. In 1994, a combination of these acute and chronic complications made diabetes mellitus the seventh leading cause of death in the United States.

The current treatment methods for diabetes have improved the average life expectancy for diabetics from less than thirty years of age in 1950 to over fifty years of age today. This last revolution in the treatment of diabetes was due primarily to the production of injectable insulin and the development of blood glucose measurement techniques. Improvements have been made in recent years with the development of implanted insulin pump devices and the knowledge that tight control of blood glucose levels can prevent sixty percent of all long-term complications of diabetes.

Currently the primary impediment to tight control of blood glucose levels is the state of the art of glucose sensing technology. All currently available glucose-sensing techniques require a sample of blood to be taken, usually from the fingertip. This sampling procedure is painful, inconvenient, and intrusive on the daily life of the patient. Furthermore, people with insulin dependent diabetes have complained that it has become difficult to obtain blood samples to test blood sugar levels after years of pricking their fingers and scar tissue buildup. Consequently, most patients do not measure their blood glucose levels as often as they should in order to prevent long term complications of the disease. Thus, the common glucose monitoring devices require high maintenance.

According to a study released by the National Institute of Health in 1993, tight glucose control has been predicted to stop sixty percent of all long-term complications of diabetes. The National Center for Disease Control estimates that there are 15.7 million Americans suffering from diabetes mellitus with almost 200,000 deaths related to diabetes in 1998. The annual market for glucose detection technology was estimated at 20 billion dollars. Current research is being performed to develop a better glucose detection system, such as non-invasive, near-infrared sensors and trans-cutaneous chemical sensors.

An improved means of collecting blood glucose measurements would be desirable to patients, doctors, and insurance companies for several reasons. From a user perspective most diabetic patients would be happy with an improved sensor simply because they would no longer be required to take blood samples several times a day. This factor alone would contribute to an improvement in the quality of life for the patient.

As taking a blood sugar measurement becomes less obtrusive, the patient would be more likely to take these measurements on a regular basis. This type of regular and frequent measurement has always been the most helpful to physicians in treating diabetes. If used in conjunction with an implanted insulin pump that allows for frequent small injections of insulin, rather that occasional large injections, blood glucose levels could be controlled to close to the normal range. This type of control would improve not only the patient's quality of life, but also their long-term health.

Because of the long-term health benefits, even a very expensive device that could produce these results should be desirable to insurance companies. A one-time investment of this type would prevent a great deal of expense later in life. However, it is also probable that an implanted glucose-measuring device could be designed that would be less expensive than current glucose measuring techniques. The average diabetic currently pays nearly $4,000 annually in diabetes related health expenses. Roughly, one quarter of that amount is for glucose measuring supplies. The total cost of glucose measuring supplies to the insurance company is higher than one thousand dollars annually. Even a several thousand dollar device would pay for itself in the course of a few years even when long term health benefits are not considered. Due to the cost of blood glucose measurement and the number of diabetics in the United States, the current estimated annual market for an implanted glucose sensor is estimated to be $20 billion in the United States alone. If such a device were sold in a global market the annual market would probably be at least doubled.

The requirements of an implanted glucose sensor were derived from knowledge of how current glucose meters work (Bayer Corp. 1998) and with a general knowledge of implanted device requirements. In order for an implanted sensor to work as well as current external glucose meters the sensor would optimally have to detect a range of blood glucose levels from less than 40 mg/dl to several hundred mg/dl. There are also several other considerations that have been suggested to be necessary for the ideal sensor to be produced. These factors include, but are not limited to, high sensitivity, low power needs, timely response, little calibration, miniaturizable, easily produced, and long-term usability (Walsh, 1997).

Several main methodologies have been proposed to date in order to produce a sensor that meets these requirements. Much research has been put into developing a chemical sensor (Armour, 1990; Cough and Armour, 1995; Lemke and Henning, 1990; Leypoldt, 1984; MiniMed, 1997; Peura, 1998; Tse and Couglt, 1987; Walsh, 1997). Most of these chemical methods are dependent on the reaction of glucose with some sort of enzyme to produce an electrically measurable change in ion concentrations. The most common method used in this type of device is the reaction of glucose and oxygen with an enzyme called glucose oxidase. This reaction produces gluconic acid and hydrogen peroxide. Platinum will then remove the hydrogen from the hydrogen peroxide and produce hydrogen gas, oxygen, and two electrons. These electrons create a current that is proportional to the concentration of glucose. The problem with this method is that if the hydrogen peroxide is not broken down and then escapes the device, it would be harmful to the body. In addition, all chemical sensors have the problem of enzyme denaturation. This problem requires frequent calibration of the sensor, and can limit the life span of the sensor greatly.

Another primary method that is being researched is the use on non-invasive optical detection methods (Heise, 1994; Robinson, Ries et al., 1992). This method relies on looking at the absorption or reflectance spectrum of tissue using electromagnetic radiation in the near infrared region. This wavelength of radiation will penetrate well into the tissue. However, it has not been shown that absorption in this region is particularly selective to glucose. The greatest difficulty has been the complexity of the light-tissue interaction. By using a non-invasive approach, this device is required to look through many different types of tissue. This method has been used successfully in clinical situations, but there is a large amount of error introduced by placing the device improperly on the body. Small changes in tissue orientation and placement have the potential to greatly affect the measured results. In addition, the high-powered light sources and sensitive detectors used are very expensive and difficult to maintain.

Past research by H. Zeller, P. Novak, and R. Landgraf provided many results that were useful in the choice of glucose sensor method (Zeller et al., 1989). Their research suggested that an infrared sensor would respond directly to the glucose molecule instead of indirectly, like electrochemical methods, and would provide the needed long-term stability required by an implanted device. Thus, Zeller et al. investigated the absorption of glucose in the mid-infrared region from 2.5 μm to 20 μm. They discovered that under physiological conditions there were five main glucose absorption peaks: 1040, 1085, 1109, 1160, and 1365 cm¹ (9.62, 9.22, 9.02, 8.62, and 7.33 μm, respectively). This region is often referred to as the “fingerprint region.” Various substances were investigated that are related to blood: hemoglobin, albumin, α-β globulin, γ-globulin, 1 m-urea solution, 2 m-glucose solution, glucose in blood, blood, and plasma. It was shown that the 1040 cm⁻¹ peak is unique to glucose. The 9.22 μm peak was also found in several substances, but the results suggested that albumin was the only substance to contribute to this absorption in the plasma. The 9.02 μm peak was found also in hemoglobin. The 1 μm-urea solution was the only substance to interfere with the 8.62 μm, but this is likely to cause problems. The 1365 cm⁻¹ peak showed several peaks for various substances. The Zeller et al. research concluded that the best peak to use is the 9.62 μm, and that the 9.02 μm and 8.62 μm peaks are useful if other substances are accounted for.

There have been several reports of using an implanted optical method of detection in the literature. A company called Sensors for Medicine has been looking for an enzyme that will fluoresce only when bonded to glucose, that would be imbedded on an implanted microchip (Walsh, 1997) There has also been some research done into using the mid-infrared region of the electromagnetic spectrum which has been shown to be sensitive to glucose absorption (Zeller, Novak, and Landgraf, 1989). However due to the short penetration depth of radiation at this frequency this method was not pursued for the non-invasive optical methods.

In order for a glucose measurement device to have the far-reaching impact described above it would have to meet several criteria. The first and most obvious criteria is that the process be painless. It would also not require a blood sample. The device would also need to provide nearly continuous measurement. In order for nearly continuous measurement to be achieved, the device would have to be very portable. With currently available technology, this means that the device should be implanted. These specifications and obvious safety concerns produce the following list of requirements that would have to be met by an ideal glucose-sensing device.

The device should fulfill the following requirements: high sensitivity, low power, fast response, safe for chronic use, does not consume what is being measured, miniaturizable, and does not require frequent calibration (Walsh, 1997). Further requirements for the present invention are that it reliably provides fast, accurate readings; not harmful to the patient; long-term stability; does not use blood samples; low power requirements; miniaturizable and comfortable; infrequent calibration requirements; will not fail without warning; can be operated by low-trained patients; possible interaction with an implanted insulin pump. What is needed, then, is a glucose monitoring system and apparatus that allows for tight control and low patient maintenance.

### SUMMARY OF THE INVENTION

A subcutaneous glucose sensor is provided which utilizes an infrared emitter which transmits light through a narrow sample of interstitial fluid held in a small sample trough. The sample trough is transparent to light at the necessary wavelength and is also highly inert. The device is implanted in the subcutaneous tissue and thus monitors interstitial fluid glucose levels, which have been shown to have a direct correlation to blood glucose levels. The present concept measures changes in absorption at mid-infrared frequencies (9-10 μm) due to glucose concentration changes. The sensor can be incorporated with an insulin pump in order to create an artificial pancreas.

One advantage of the present invention is that the best glucose measurement is one that allows the progress of diabetes complications to be checked, and a self-controlled implanted device minimizes patient maintenance. This device that can improve the lives of millions of diabetics around the world, by increasing glucose monitoring intervals and control, and reducing the pain and numbing effects of the current monitoring methods.

Other objects, features, and advantages of the present invention will become apparent upon reading the following detailed description of embodiments of the invention, when taken in conjunction with the accompanying drawings and the appended claims.

### BRIEF DESCRIPTION OF THE DRAWINGS

The invention is illustrated in the drawings in which like reference characters designate the same or similar parts throughout the figures of which:

FIG. 1 is a top schematic view of an apparatus according to a preferred embodiment of the present invention.

FIG. 2 is a side schematic view of an apparatus according to a preferred embodiment of the present invention.

FIGS. 3A and 3B are schematic views of the sample trough or well.

FIG. 4 is a graph of the light wavelength absorption.

FIG. 5 is a graph of the different attenuation wavelengths for various substances.

### DESCRIPTION OF THE PREFERRED EMBODIMENTS

The operating parameters for the present invention define the design specifics of the device to a large extent. These parameters are defined by the location of the device within the body and the need for accurate and precise glucose measurement. Because the device is an implant it must be suitable for contact with the soft tissues within the body. The device must operate at body temperature, must not cause bleeding or scarring, and must not cause any great discomfort to the user. In addition the device must not allow itself to be harmed by the body, or must at the very least recognize when the body's response will prevent proper performance. The primary concern in this area is the fouling of the sample area with proteins.

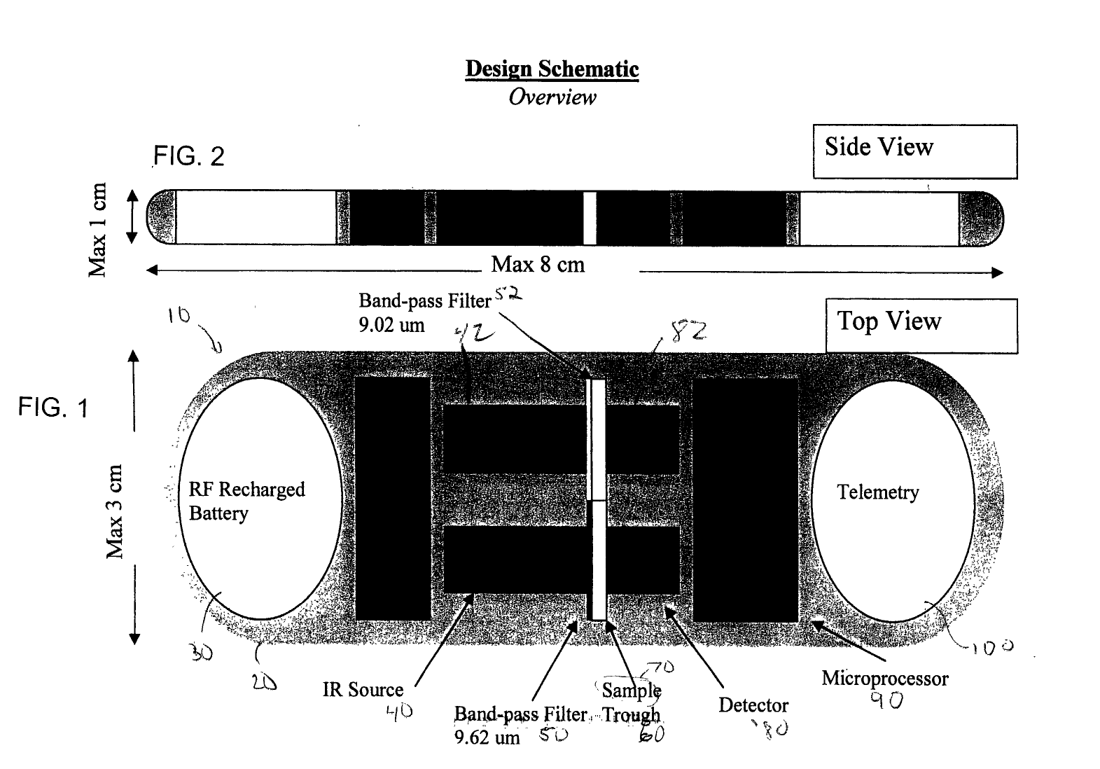

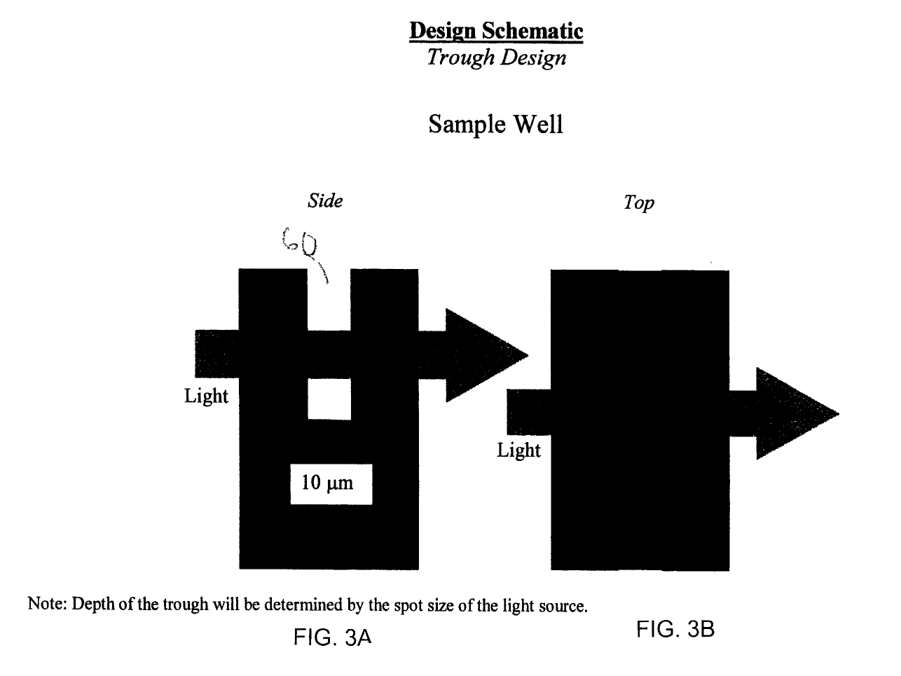

In its most general embodiment, shown in FIGS. 1-3, a device 10 comprises a housing 20, which is preferably biologically inert when used in a patient's body. A power source 30 is preferably a battery which is preferably rechargeable. At least one, and preferably two energy sources 40 and 42, preferably a light emitting diode (“LED”) or laser diode draws power from the power source. It is to be understood that other energy sources are contemplated as being within the scope of the present invention. The energy source preferably produces energy in the infrared region. At least one, and preferably two, band pass filters 50 and 52 filter energy from the energy sources 40 and 42 so that at least one, and preferably two, different wavelengths of energy are passed through the filters and to the sample 70. A sample trough 60 which can hold a sample 70 of fluid (not shown) is defined in the housing 20. At least one, and preferably two, light detectors 80 and 82 are positioned to receive transmitted energy passing through the sample trough 60. A microprocessor 90 can process the information received by the detectors 80 and 82. A telemetry device 100, associated with the device 10, functions as described hereinbelow.

The device 10 measures a range of glucose concentrations from about 20 mg/dl to about 600 mg/dl (Bayer, 1998). The accuracy of the measurements produced must be correct to within about 10 mg/dl of the actual value to compare to currently available glucose meters, although a closer range of +/−2 mg/dl is more desirable. This would require a test-retest precision that is considerably better than about 10 mg/dl. In order to ensure this accuracy, two redundant systems using different wavelengths of radiation are used in conjunction.

A schematic of a device 10 of the present invention is included in FIGS. 1-3. The dimensions of 8 cm by 3 cm by 1 cm are the currently generally accepted maximum allowable size for a subcutaneous implant (Herline, 1999). It is to be understood that larger or other shaped devices may be used by adapting current technology known to those skilled in the art and by designing the housing for use in a particular location in the body. Because the present technology contains no moving parts, most of this design could eventually be miniaturized to the size of a microchip. FIGS. 1-3 also show one configuration for the necessary components. One of ordinary skill in the art can modify the configuration for use with particular components. It is also possible that some of the components can be in a housing that is subcutaneous and some of the components can be in an external device in proximity to the subcutaneous housing, with communication between the two being achievable by radio frequency (RF) or other data communication medium known to those skilled in the art.

The function of the device components can be mapped by following the power flow through the device. The beginning for this flow is the rechargeable battery 30. Pre-processor circuitry then controls the power flow to the light sources 40 and 42. The light sources 40 and 42 convert the stored electrical energy to usable electromagnetic radiation at the specified frequency. This light radiation then passes through the interstitial fluid sample 70 where it is attenuated according to the concentration of glucose. The remaining light that passes all the way through the sample 70 is measured in the optical detector 80. The ratio of light detected to light emitted for both wavelengths is then passed to the post-processor where this data is fit to a mathematical model to determine the glucose concentration. This information is then passed out of the device 10 by the telemetry system 100.

The battery 20 is necessarily a high-tech component of the device 10. The style of battery which can preferably be used is already in use in pacemakers where a long term battery with a small size is needed. In order to accomplish this, the battery 20 can be recharged using an RF signal or by other signals known to those skilled in the art. This means that the battery can be recharged without removing the device and without puncturing the skin.

In order to conserve battery power the light sources must be efficient. An example of an efficient light source is a diode laser that will operate at the desired wavelength of light radiation and at body temperature. A light emitting diode can also be used when coupled with a narrow band pass filter. This type of light source can easily operate at body temperature, and can be configured for a broad range of frequencies before implantation. However, the power efficiency suffers greatly compared to a laser diode. The LED is preferably filtered to attain the needed frequency.

The light beams produced by the energy sources 40 and 42 are passed through a sample 70 of interstitial fluid that is contained in a sample trough 60. The trough 60 is open to receive interstitial fluid on the top of the trough. The width of the trough 60 is preferably set at 10 μm to allow for the maximum dynamic range of the signal. A different width trough 60 may be used when using a signal having a greater or lesser maximum dynamic range.

The calculations associated with this embodiment are reviewed in the following discussion. This discussion covers the optical concepts regarding absorption and Beers law that were utilized in the design of the present invention. When light interacts with a molecule it can do one of three things. It can be absorbed and re-radiated, transmitted; it can be scattered, deflected; or it can be absorbed. During absorption the energy from the incident photon is transferred to the molecule. Normally this energy becomes heat, although sometimes it can be used to drive a chemical reaction as in the case of photosynthesis.

In a non-scattering, absorbing medium the amount of light at any point along the light beam's path length will decrease with position. This decay is exponential in nature and follows Beer's Law.

Eₓ=Eₒe^{(-∀x)}

Where Eₓ is the light energy at any position x along the light beam. Eₒ is the initial light energy incident on the medium and alpha, (a), is the linear attenuation coefficient.

The linear attenuation coefficient alpha is dependent on the concentration of the absorbing molecule in the medium. So that A=kc and the two are directly proportional. Therefore, as the concentration of the absorbing molecule increases the amount of light penetrating the medium to a given depth x will decrease exponentially. Over a small change in concentration it may be reasonable to approximate this decrease as a linear relationship.

The following calculations show the derivation of the sample path length. The definition of transmission is

T=Iₜ/Iₒ

Where T is transmission, Iₒ is the incident light and Iₜ is the transmitted light. This is then used with Beer's Law:

l=1ₒ*(eˆ (−a*1))

Where alpha, (a), is the attenuation coefficient and l is the path length. By substituting transmission into Beer's Law, the result is:

T=(eˆ (−al))

This can then be used in the formula for the power of the detected signal:

Pₛ=PₒT

Where PS is the detected signal power and PO is the initial power. The dynamic range of the signal as glucose concentration changes is then

DR=(PₒTₘₐₓ)−(PₒTₘᵢₙ)

Where DR is the dynamic range, Tₘₐₓ is the maximum transmittance with low glucose concentration and Tₘᵢₙ is the minimum transmission at high glucose concentration. By substituting the previous formula, one gets:

DR=Pₒ(e^{-alowcl}−e^{-bahighcl})

Where DR is the dynamic range, alowc is the attenuation coefficient at low glucose concentration and ahighc is the attenuation at high glucose concentration. Substituting in the experimental values for the attenuation coefficients and solving for the maximum dynamic range the optimal path length is found to be 10 μm.

l=10 μm

An additional factor to be considered is the Fresnell reflection. When any wave propagation of energy crosses from a medium of one impedance to a medium of a different impedance some of the wave is reflected and some of the wave is refracted. The portions of the wave that are parallel to the incident surface and the portions that are perpendicular to the incident surface behave differently so that there are two coefficients describing the reflection off the impedance mismatch.

When the light beam is orthogonal to the impedance mismatch the total reflectance coefficient simplifies to the following equation.

R=((n₁−n₂)/(n₁+n₂))²

There is also an angle known as Brewster's angle where all of the light polarized perpendicular to the interface will be transmitted despite the impedance mismatch. This fact can be used to reduce the loss of power due to reflect at impedance mismatches.

Therefore, the depth of the trough 60 is determined by the spot size of the light beam. It is anticipated that diffusion will be sufficient to equalize glucose concentration within the sample area to that of the surrounding tissue. The substance that the sample trough 60 is made of will should be optically transparent to light in the region of interest. It must also be biologically inert. The two materials that have been highly considered for this application are sapphire and CVD diamond films. Glass or quartz of sufficient transparency and quality may be usable. The CVD diamond films have been chosen because they are easily produced compared to sapphire optical components. The trough volume has a practicable upper path length limit in that if too much fluid, e.g., water, is sampled, the high volume water may absorb the mid-IR light.

After light passes through the sample trough 60 it is measured by the detector 80. The primary limitation for this component is the ability to operate at physiological temperatures. This requirement led to the selection of a pyroelectric detector for this component. This type of detector absorbs pulsed light energy and measures the corresponding rapid temperature increase. Because the detector 80 is based on temperature differentials, it is very stable at biological temperatures and not affected by the gradual changes in body temperature.

The post-processor then interprets the output of the light detectors 80 and 82. The detectors have an analog voltage. This voltage is used to determine how much glucose is present in the sample aliquot. There is an inverse relationship between voltage and the concentration of glucose. The ratio of measured transmitted light to the light produced provides a value for the change in the transmission of light. This value can be correlated to glucose concentrations. In order to utilize the redundancy built into the system by including two wavelengths of light in separate detectors 80 and 82 the recorded transmission values for the two wavelengths can be reconciled using a microprocessor 90 that can recognize a wide range of inputs and properly deal with novel inputs that fall within a reasonable range.

A possible processing solution would be to use a neural networks application. This method was tested using arbitrary absorption ratios that are linearly proportional to a change in glucose concentration. Simple perceptrons were used to insure that the two wavelength absorption ratios were relatively close together. This would warn of failure if these two ratios are significantly different. The perceptrons worked moderately well and could be enhanced, but other comparators are possible to solve this problem.

The translation of the absorption ratios into glucose concentrations is linear. However, the neural network needs to approximate the function in a manner that allows for generalization between trained values. Back-propagation performs function approximation well as does radial basis function networks. The function was attempted with back-propagation, but the results were unsatisfactory with little convergence resulting. The radial basis function networks did converge and will give workable results. Initial conclusions show that this method can be used, but with 120 neurons for each network, the networks are larger than desired and should be modified if neural networks are to be used.

As a final safety precaution a telemetry system 100 is preferably built into the device to allow testing of the power system and device, calibration, and warnings when the device fails. A proper fail-safe mechanism for such a system requires a periodic signal and test from the device to ensure that it is working properly. If this signal is not received, an alarm is sounded on a companion pager style device 110 (not shown). This device 110 or other remote signaling device may also be used to read out the measurements obtained from the sensor. Such a signaling device 110 should be capable of receiving a signal transmitted by the device 10 and transmitting a detectable signal, such as, but not limited to, audible, display, vibration, combinations of these, or the like.

The final calculated glucose concentration is transmitted to a receiver 110, such as a pager device using spread-spectrum telemetry or other devices known to those skilled in the art. The information is coded and put on the correct frequency. Only the pager or other receiving device 110 has the correct key to interpret the information and it will not interpret other signals as good information without that key. This sort of telemetry allows for a low power signal to be used without concern for signal interruption. The technology necessary for this component has been produced at the Oak Ridge National Laboratories and has been placed on a single miniaturized microchip, with the exception of the antenna.

The entire device 10 is then encased in a bio-compatible sheathing or housing. This sheathing may be a Teflon® or other biologically inert, nondegradable coating similar to the material used to encase pacemakers. A new surfactant coating is currently under development at Case Western Reserve University that would improve the bio-compatibility of the device by making the surface of the device appear more like a cell membrane to the body (Ruegsegger, 1999). This type of surfactant coating could also line the sample trough to prevent fouling of the sample area with undesired proteins.

Safety concerns for the device 10 incorporate both those concerns normally associated with any type of implanted device and those normally associated with medical information systems. As an implanted device, it is critical that the device of the present invention not cause any harm to the body. This could be through toxicity, thermal damage, or through rejection. Toxicity is not a probable threat with an implanted device because no toxic substances are in contact with bodily fluids as in implanted chemical sensors. Whenever using an absorption based light-tissue interaction, thermal damage is a possibility. In this case the threat of thermal damage is greatly reduce by using low powered light sources and a small sample volume. The device 10 has also been designed so that the surface of the device 10 will remain at physiological temperature.

The more pressing area of safety concern deals with typical issues of medical information systems. Improper information can lead to an improper diagnosis. This is the reason for the redundancy designed into the present invention. By comparing two separate measurements the accuracy of the device and robustness of the measurement is greatly increased.

In the case of the present invention, there is also a need to warn the user before information from the sensor becomes unavailable. The user may become reliant on warnings from the sensor when their glucose levels are becoming too high or too low. If the device were to shut off without warning the user may fall into diabetic shock. This is the most significant safety issue. To provide a safeguard the telemetry system 100 of the device 10 can communicate with a pager-type device 110 that is carried with the user. This pager 110 can be used for reading glucose levels, and can also perform the function of self-testing the device 10. This could be performed by querying the device 10 and requiring an appropriate response. If the response is not received, an alarm could be sounded. This type of interaction could also be used for calibration of the sensor.

This embodiment provides a solution to the problem of providing a long term implanted glucose sensor. The combination of mid-infrared optical detection and an implanted sensor provide a sensor with unique beneficial aspects compared to other sensors under development. The two primary concerns currently associated with this device are the power concerns and the bio-compatibility issues. The long term power required to drive an LED light source can be more than can reasonably be expected from existing pacemaker style batteries. Development of an appropriate laser diode would be the greatest single improvement that could be made to alleviate this problem. However, other techniques may be available to improve the efficiency of the LED source system, such as finding the narrowest band source possible. It may also be possible to increase the power of the battery.

The biocompatability issue of concern deals with the fouling of the sample trough 60 with proteins. This would prevent interstitial fluid from diffusing into the sample trough 60. The use of the surfactant coating and CVD diamond may be enough to prevent fouling of the sample trough 60, but this has not been tested. An alternate method for avoiding this problem may be to introduce moving parts to the design and have the sensor clear the sample area with a small plunger of some sort.

In an alternative embodiment of the present invention is the incorporation of the device as a feedback mechanism in conjunction with and for an implanted insulin pump 200 (not shown). This provides a patient with a glucose measuring and insulin delivery system for controlling blood glucose levels. The combined system can provide insulin control that would allow the return of near normal functioning of the body. An insulin delivery apparatus as known in the art can comprise: (1) a reservoir capable of holding an amount of insulin, (2) means for delivering said insulin from said reservoir to a patient, said means comprising a pump, and (3) an insulin delivery processor for determining when to actuate said pump to deliver an amount of insulin.

Even if the device of the present invention is not used in conjunction with an insulin pump, it still provides a dramatic improvement over current methods. The patient cannot forget to take a blood measurement with this device, since measurement is automatic. In addition the measurement will be completely unobtrusive and painless once the implant is installed. This implant would also allow for measurement during periods of sleep and allow for early warning when glucose levels are heading toward dangerous levels.

The present invention also provides a method of detecting and measuring a component of a fluid, comprising the steps of (1) providing a device comprising a housing, a power supply, a processor, at least one energy source, at least one detector capable of detecting the energy emitted by the energy source, and at least one sample trough associated with the housing capable of retaining an effective amount of the fluid; (2) causing an aliquot of fluid to be retained within the trough; (3) emitting energy from the energy source so as to impinge on the fluid in the trough; (4) detecting the energy passing through the fluid in the trough; (5) transmitting an output signal from the detector to the processor; (6) interpreting the output signal; (7) determining the presence and/or concentration of the component in the fluid aliquot, and optionally (8) transmitting data containing the calculated concentration to a remote device.

The present invention can also be used to measure impurities in industrial applications. High temperature applications could be used, because the CVD diamond will work at high temperatures. The Teflon® coat may not be optimal for use above the Teflon® material softening or melting point temperature and that would have to change. Any water-based fluid will need to have a small path length, because water absorb highly in the mid-IR region. The peaks, like with the present invention, are much higher than the water, but too much water means all light absorbed.

Other fluids which can be measured need to have peaks that are not influenced by other changes in the environment. Hemoglobin, plasma, tears, semen, urine, other body fluids or semi-fluids and the like are possible body fluids which could have components measured according to the present invention. The present invention can also be used to detect and measure the presence and amount of particular proteins, amino acids, nucleotides, and the like that may increase concentration prior to the return of cancer. If the implantable device of the present were implanted after cancer the first time, then it could catch the spike in protein production associated with the return and get the cancer attacked again before any other conventional forms of detection have found the cancer returning. The present invention could also be used on a probe instead of an implant, as a better technique for biopsy.

Although only a few exemplary embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention as defined in the following claims. It should further be noted that any patents, applications or publications referred to herein are incorporated by reference in their entirety.

The invention will be further described in connection with the following Examples, which are set forth for purposes of illustration only. Parts and percentages appearing in such Examples are by weight unless otherwise stipulated.

### EXAMPLES

The sensing method utilizes glucose modification. This sensing method was verified by experimentation. A first experiment used a concentration of glucose that was two orders of magnitude greater than physiological levels. This experiment was then modified to test for a measurable change in absorption due to glucose concentration changes at physiological levels.

The next experiment was conducted using a Bruker™ IFS 66 v to measure the reflectance spectrum of various samples. The samples measured were of plasma at varying glucose concentration and several aqueous glucose solutions of varying concentration. The glucose concentrations used were all within the normal physiological range with the exception of one sample of higher concentration that was used to compare to the previous experiment.

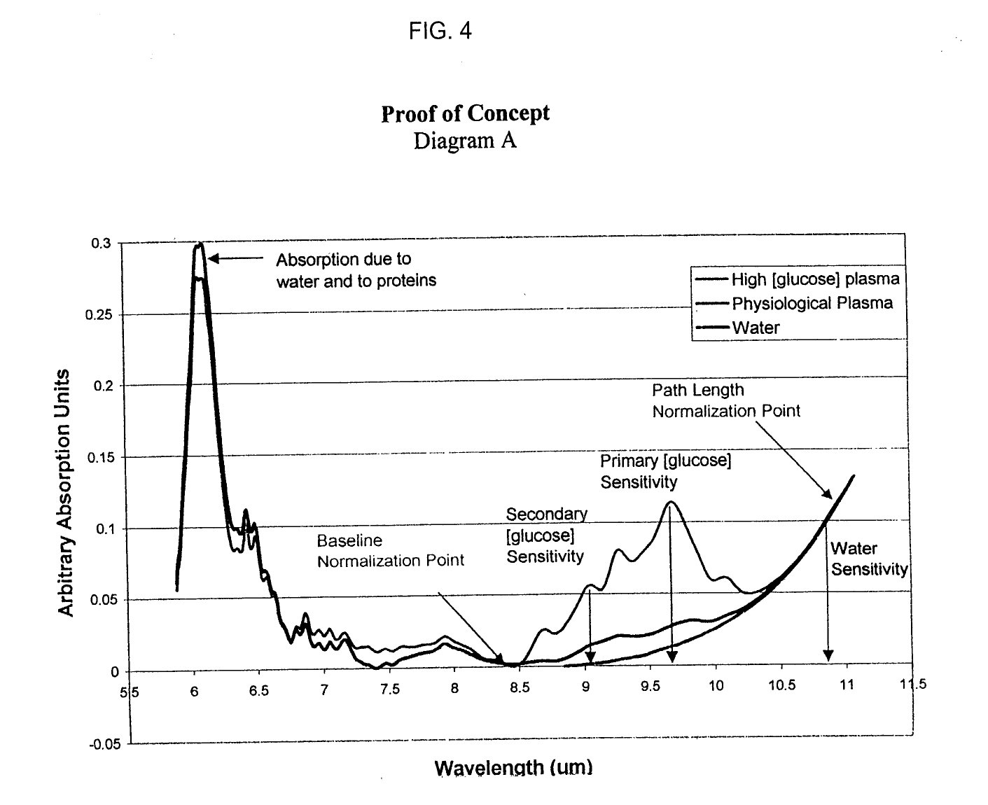

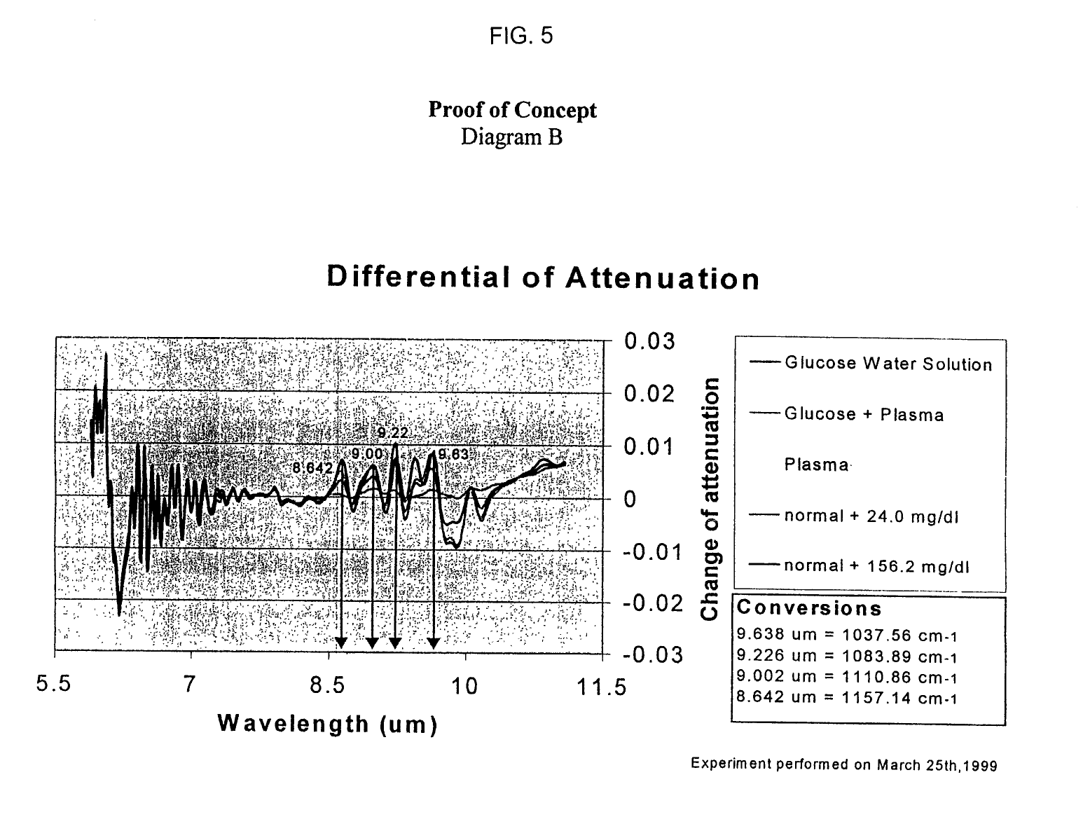

This resulted in proof of the concept of using absorption in the mid-infrared range to measure changes in glucose concentration. There was a definite linear increase in absorption in the 8.5 to 10.5 μm wavelength range with increasing glucose concentrations. FIG. 4 shows the increase in absorption for the primary (9.62 μm) and secondary (9.02 μm) peaks. These measurements were taken at room temperature (˜25° Celsius), not body temperature, so the water absorption seen in the diagram will shift more to the right when implanted in the body. This is a result of the hydrogen bonding in the water, which is stronger at colder temperatures. The linear increase in absorption is not shown on the diagram but was evident in the results. Differential changes in glucose concentration were measurable by observing the increase in absorption relative to the base-line curve. The differential method shown in FIG. 5 showed a better correlation to changes in glucose, especially smaller changes, with an R² equal to 0.98. Glucose concentrations of 2.4 mg/dl were distinguishable with this method.

Using the FIFTIR machine, we were able to have similar results to Zeller et al. and make similar conclusions. The following major absorption at the wavelengths of 9.62, 9.02, 9.26, and 10.1 μm were observed. The wavelengths for glucose absorption that were chosen as the most significant and most usable were 9.02 μm, 9.26 μm and 9.62 μm. The 9.62 μm peak had the strongest absorption and when compared to the Zeller et al. paper was not present in blood either. The 9.26 μm peak had the next largest peak, but this peak is also found in other blood proteins, hemoglobin and albumin (Zeller et al., 1989). The 9.02 μm was smaller than the previous two peaks but μsable. This peak too can be found in hemoglobin (Zeller et al., 1989). However, as mentioned above, the design of the present invention will measure the interstitial fluid, which is closely related to plasma, and not blood. The plasma did not have the absorption peaks of hemoglobin, so the 9.02 μm wavelength can be used as well (Zeller et al., 1989). The 9.26 μm wavelength is still hindered by the absorption interference of albumin even with hemoglobin accounted for. If it is shown that the albumin is relatively constant, or does not significantly effect the level of absorption, then the 9.26 μm peak may also be considered. Only two peaks are needed for the preferred embodiment, so the 9.02 μm and the 9.62 μm are used.

What is claimed is:

1. A device for sensing the presence or concentration of a component in a fluid, comprising:
a. a power supply;
b. a processor;
c. at least one energy source;
d. at least one detector capable of detecting energy emitted by said energy source;
e. at least one sample trough capable of retaining an aliquot of fluid; and,
f. a housing.

2. The device of claim 1, further comprising a telemetry device associated with said processor and capable of transmitting a signal from said processor to a remote receiving means.

3. The device of claim 2, wherein said remote receiving means is a pager capable of detecting a signal from said telemetry device.

4. The device of claim 3, wherein said signal transmitted from said telemetry device is an alarm signal transmitted in response to failure of said device to obtain measurements.

5. The device of claim 1, wherein said signal contains measurement data from said detector.

6. The device of claim 1, wherein said energy is light.

7. The device of claim 1, wherein said energy source is a light emitting diode.

8. The device of claim 1, wherein said energy source is a laser diode.

9. The device of claim 1, wherein said energy source generates energy in the near infrared range.

10. The device of claim 1, wherein said energy source further comprises a filter.

11. The device of claim 10, wherein said filter comprises a narrow bandwidth pass filter.

12. The device of claim 1, wherein said energy source provides energy at least two detectably distinct wavelengths.

13. The device of claim 1, wherein said detector is capable of detecting transmitted energy in the near infrared range.

14. The device of claim 1, wherein said detector is a pyroelectric detector.

15. The device of claim 1, further comprising a second detector capable of detecting a distinct wavelength of energy.

16. The device of claim 1, wherein said power supply is a battery.

17. The device of claim 1, wherein said battery is rechargeable.

18. The device of claim 1, wherein said trough is generally U-shaped and is defined in said housing.

19. The device of claim 1, wherein said trough is coated with a surfactant.

20. The device of claim 4, wherein said light has a wavelength of the mid-infrared.

21. The device of claim 4, wherein said light has a wavelength of from about 9 μm to about 10 μm.

22. The device of claim 1, wherein said component is glucose.

23. The device of claim 1, wherein said fluid is hemoglobin, plasma, tears, semen, or urine.

24. An apparatus for delivering insulin and sensing the presence or concentration of glucose or other component in a fluid, comprising:
a. a power supply;
b. a processor;
c. at least one energy source;
d. at least one detector capable of detecting energy emitted by said energy source;
e. at least one sample trough capable of retaining an aliquot of fluid;
f. a housing; and,
g. an insulin delivery apparatus comprising
i) a reservoir capable of holding an amount of insulin,
ii) means for delivering said insulin from said reservoir to a patient, said means comprising a pump, and
iii) an insulin delivery processor for determining when to actuate said pump to deliver an amount of insulin,
such that said insulin delivery processor is in communication with said device processor to provide a feedback loop between said sensing device and said delivery apparatus to maintain proper insulin level.

25. A method of detecting and measuring a component of a fluid, comprising the steps of:
a. providing a device comprising
iv) a housing,
v) a power supply,
vi) a processor,
vii) at least one energy source,
viii) at least one detector capable of detecting the energy emitted by the energy source, and
ix) at least one sample trough associated with the housing capable of retaining an effective amount of the fluid;
b. causing an aliquot of fluid to be retained within the trough;
c. emitting energy from the energy source so as to impinge on the fluid in the trough;
d. detecting the energy passing through the fluid in the trough;
e. transmitting an output signal from the detector to the processor;
f. interpreting the output signal;
g. determining the presence or concentration of the component in the fluid aliquot.

26. The method of claim 25, further comprising a step h. transmitting data containing the calculated concentration to a remote device.
